(19) United States
(12) Patent Application Publication
(10) Pub. No.: US 2022/0118237 A1
(43) Pub. Date: Apr. 21, 2022

(54) DEVICES FOR TOPICAL DELIVERY OF ACTIVE AGENTS TO A TARGET SITE

(71) Applicant: CANNABIDOSE MEDICAL LTD., Kfar Saba (IL)

(72) Inventors: Sagi SLUTSKI, Kfar Saba (IL); Amnon SLUTSKI, Kfar Saba (IL)

(73) Assignee: CANNABIDOSE MEDICAL LTD., Kfar Saba (IL)

(21) Appl. No.: 17/298,358

(22) PCT Filed: Nov. 29, 2018
(86) PCT No.: PCT/IL2018/051305

§ 371 Date: May 28, 2021

(51) Int. Cl. A61M 35/00; A61M 37/00

(52) U.S. Cl. CPC A61M 35/10; A61M 2202/0007; A61M 2205/04; A61M 37/00

(57) ABSTRACT

Provided are devices, i.e. medical devices, for topical delivery of various active agents to a target site. More specifically, provided are devices for controlled release of an active agent to a skin portion upon contact with an aqueous fluid, such as perspiration, exudate or external applied water-based fluids.

### TECHNOLOGICAL FIELD

The present disclosure concerns devices, i.e. medical devices, for topical delivery of various active agents to a target site. More specifically, this disclosure concerns devices for controlled release of an active agent to a skin portion upon contact with an aqueous fluid, such as perspiration, exudate, lacrimal fluid, external applied water-based fluids, etc.

### BACKGROUND ART

References considered to be relevant as background to the presently disclosed subject matter are listed below:

US 2006/173430

WO 08/059266

U.S. Pat. No. 5,141,750

Acknowledgement of the above references herein is not to be inferred as meaning that these are in any way relevant to the patentability of the presently disclosed subject matter.

### BACKGROUND

Devices for topical delivery of various active and non-active agents are known. Most such devices are based on layered structures in which one of the layers contain the agent, typically a polymeric film into which the agent is solubilized or embedded. In another configuration, the device may be in the form of a pouch that contains the agent, that is released to the target site upon breach of the pouch. Such devices often have a limited flexibility and hence provide limited contact area with the target site. In addition, such devices often have limited capacity to carry and deliver a plurality of active agents or a plurality of doses.

As such devices are typically designed for self-application by the user or, pending the need by care provides, e.g. physicians, these devices also require to be designed for ease of application to the site to be treated or to the site of application and simple to use.

### GENERAL DESCRIPTION

The present disclosure concerns devices, e.g. medical devices, that are designed to topically deliver one or more active agents to a target site in a controlled and selective manner, as well as provide a sequence of delivery of the active agent once the device is brought into contact with an aqueous liquid, such as perspiration, an exudate, lacrimal fluid, etc. In some designs of the devices of this disclosure, the device also has improved flexibility to maximize the contact area with the application site. The devices of this disclosure are designed to selectively control the timing and location of delivery of the active agent by providing, inter alia, a highly controllable local disintegration of one or more portions of the device in order to release the active agent contained therein in a targeted and timed manner (e.g. substantially immediate and local release of active agent from the device to the target site upon contact of the device with the target site, or controlled and/or timed release of the active agent that depends on the structural features of the device). Further, the devices of this disclosure are designed to release the active agent therefrom to the target site only upon contact with an aqueous fluid, e.g. perspiration, exudate, other body liquids or externally-applied water-based fluids, thus enabling release of the active agent to, e.g., skin burn areas, ulcers, or upon conditions causing perspiration (e.g. increase in body temperature, cold perspiration, etc.).

For this purpose, the present disclosure provides medical devices for topical application, for example bandages or dressings, that comprise a plurality of cells that contain at least one active agent, and are configured for selective release of the active agent to the target site upon contact with an aqueous (water-based) bodily fluid, i.e. perspiration or exudate (or even lacrimal fluid) or by applying water-based fluids onto the device during and/or after its application to the skin. By segmenting the devices to a plurality of cells, improved flexibility of the device is obtained, as well as isolation of various active agents one from the other prior to use. Segmentation further enables design of each cell according to the desired timing of disintegration, such that control over the timing of release and/or dosing of the active agent may be obtained.

Thus, in a first aspect of this disclosure, there is provided a flexible device for topical delivery of at least one active agent to a target site, the device comprises a flexible substrate for placing onto a skin portion, and having a plurality of spaced-apart cells, each cell containing the at least one active agent and having at least one wall portion made of a film of at least one polymeric material that is at least partially disintegrable upon contact with an aqueous fluid, i.e. perspiration or exudate or lacrimal fluid or externally applied water-based fluid, to thereby release the active ingredient to the target site.

The term flexible or any lingual variation thereof, is meant to denote the property of pliability, being able to bend or fold without applying significant force while maintaining structural integrity. In the device of this disclosure, flexibility is rendered possible by the combination of a flexible substrate and the segmentation of the agent-carrying cells carried by the substrate. Segmentation to a plurality of spaced-apart cells allows the device to be folded or bent along the division lines separating between the cells, such that the device can be formed to closely follow the contours of the body part to which the device is applied and remain in contact therewith.

The cells carried by the substrate are closed cells, each being of a desired volume and shape. The cells may each have identical volume and/or shape, or may differ one from the other in one or both of their volume and shape. The cells are said to be spaced-apart one from the other, i.e. being distanced one from the other in a plane defined by the flexible substrate.

The plurality of cells include at least 2 cells. In some embodiments, the device includes at least 2, 3, 4, 5, 6, 7, 8, 9, 10, 12, 14, 16, 18, 20 or more cells, arranged in a spaced-apart manner on the substrate. The arrangement may be in an ordered form (i.e. an ordered array) or in random distribution.

At least a portion of the cells, at times each of the cells, contain therein at least one active agent to be topically delivered by the device to a target site. For this purpose, the cells are made of at least one polymeric material that is at least partially disintegrable upon contact with a fluid, i.e. perspiration, exudate, lacrimal fluid or externally applied water-based fluid. The term disintegrable means to denote physical or chemical destruction of the polymeric material caused by contact of the polymer with said liquid, thus causing rupture/disintegration of the cell and release of the active material contained therein. The term means to encompass any type of physical or chemical disintegration, e.g. solubilization, dispersion, chemical reaction that causes disintegration of the polymer chains, swelling or gelling of the polymer, or any other suitable deterioration of the structural integrity of the cell. The polymeric material is at least partially disintegrable, meaning that complete disintegration of the polymeric material is not compulsory; the polymeric material needs to be disintegrable to the extent that the release of the active agent contained within the cell is enabled.

Each of the cells has at least one wall portion, that is designed to come into contact with the fluid, and is made of said at least partially disintegrable polymeric film. The team at least one wall portion means to denote a wall section of the cell, typically a section of the cell's wall that is designed to come into contact with the site of application, and hence with the fluid that exists on the site of application; however, the wall portion can be any other wall section of the cell that may come into contact with the fluid. In some embodiments, the wall portion may be the majority of the cell or even the entire cell.

In the device of this disclosure, the cells may be configured for selective disintegration upon contact with said fluid. Namely, cells which have not been in contact with an aqueous fluid, such as perspiration or exudate, will remain intact. Thus, when applied to the application site, only cells which are in contact with the aqueous fluid (namely only cells which come into contact with perspiration, exudate, lacrimal fluid, externally applied water-based fluids) present on the skin portion onto which the device is applied will be disintegrated to release the active agent to the desired target site; adjacent cells will not be ruptured. Contrary to devices in which a single pouch is used and its entire content is delivered, the selective disintegration enables targeted topical delivery of the active agent(s) only to the desired target site (e.g. a burn or an ulcer) and only when conditions for such delivery are fulfilled (i.e. perspiration or exertion of an exudate from the skin portion or external application of water-based fluids to which the device is applied).

It is noted that the terms aqueous fluid and water-based fluid will be used herein interchangeably.

In some embodiments, the polymeric material is fully disintegrable within between about 5 second and 30 minutes from contact with said fluid. In other embodiments, said polymeric material may be fully disintegrable within between about 5 second and 10 minutes from contact with said fluid. In some other embodiments, said polymeric material is fully disintegrable within between about 5 second and 2 minutes from contact with said fluid.

The cells may also differ by their disintegration rates; namely, in some embodiments at least one portion of cells and at least another portion of cells are configured to have different disintegration rates to release said active agents therefrom at different rates or at different timing. In other words, some of the cells may be made of a polymeric material, or may be structured as will be described below, to have a first disintegration rate, while other cells may be configured to have a slower disintegration rate. Such an arrangement enables the formation of a release sequence of the active agent(s) from the device, for example a first active agent is released, followed by the release of another dose of said first active agent or the release of a second active agent. Such arrangements will be described in more details below.

The cells may also differ in their disintegration rate, such that the device is configured for controlled release of a plurality (i.e. two or more) doses of the active agent from the device. Namely, the cells can be configured to disintegrate at different rates, such a sequence of doses of the active agent can be delivered to the user, each cell comprising one dose of active agent to be administered—the time intervals between the doses being determined by the difference in the disintegration rate of the cells. The appropriate dosage may vary according to such parameters as the therapeutically/cosmetically effective dosage to be administered as dictated by and directly dependent on the individual being treated, the unique characteristics of the active agent and the particular therapeutic or cosmetic effect to be achieved.

In the context of the present disclosure, the term polymeric material (or polymer) includes homopolymers, copolymers, such as for example, block, graft, random and alternating copolymers as well as terpolymers, further including their derivatives, combinations and blends thereof. In addition to the above the term includes all geometrical configurations of such structures including linear, block, graft, random, alternating, branched structures, and combination thereof. Block copolymer is meant to encompass a polymer formed from two or more homo-polymer subunits (blocks) linearly linked by chemical bonds (i.e. the blocks are connected end-to-end). Block copolymers with two, three, four and multiple homo-polymer units are referred to as di-block, tri-block, tetra-blocks and multi-blocks respectively. The number of monomer types in a block co-polymer may be less than or equal to the number of blocks. Thus, an ABC linear tri-block consists of three monomer types, whereas an ABA linear tri-block consists of two monomer types.

In some embodiments, the polymeric material is biocompatible (and/or its degradation products are biocompatible).

As noted above, the polymeric material is selected to be at least partially disintegrable when in contact with an aqueous fluid (i.e. perspiration, exudate, lacrimal fluid, externally applied water-based fluids, etc.) present at (or locally applied onto) the skin portion onto which the device is applied. The term perspiration refers to any fluid secreted by sweat glands onto a skin portion; the term exudate means to denote any fluid secreted or released by a wound, a sore, a skin burn, etc. of a patient. The externally applied fluid can be any fluid that comprises water as a main component.

As a man of the art would appreciate, water is one of the primary constituents of bodily fluids, e.g. perspiration, exudate or lacrimal fluid. Therefore, in some embodiments, the polymeric material is selected to be at least partially disintegrable when in contact with water, i.e. being water-soluble or water-dispersible. Typically, such polymers contain hydrophilic groups as substituents or incorporated into the backbone of the polymer chain. Examples of such water-disintegrable polymers are polysaccharide, which may or may not have a plurality of identical monomers (such as in the case of dextran) or different monomers (such as in the case of arabinogalactan). The polysaccharide may be natural or synthetic and may be branched or linear. The polysaccharide may be a chemically modified or a semi-synthetic polysaccharide, permitting association with the at least one drug moiety. Exemplary polysaccharides are starch, glycogen, cellulose, dextran, pullulan, chitosan, arabinin, arabinogalactan, galactan, galactomannan, gelatin, pectin, amilo-pectin, glycan, poly-mannan, hyaluronic acid, guar gum and any other poly sugar or synthetic combination thereof.

Other exemplary water-disintegrable polymeric materials may be comprise a polymer selected from polyethyleneoxide (PEO), polyvinyl-pyrrolidone (PVP), polyvinyl-alcohol (PVA), polyacrylic acid (PAA), polyacryloamides, polyoxazoline, cellulose ethers (e.g. HPMC, HPC), etc.

It is of note that the cells are made of said polymeric material, being in film form. Namely, the cells are typically formed out of sheets of polymeric material. The film may be a single-layered film made of said polymeric material. Alternatively, the film may comprise two or more layers of polymeric material, the layers may be the same or different in their composition, thickness, uniformity, water disintegrability, etc., as long as the multi-layered film is at least partially disintegrable to permit release of the active agent from the cell or capsule. It is noted that typically the film itself does not contain an active agent; however, in some cases, the film may contain a secondary active agent or an adjuvant to be released together with the primary active agent contained in the volume of the cell.

The polymeric material may have, by some embodiments, a molecular weight of at least about 50,000 g/mole. In other embodiments, the molecular weight of the polymeric material may be in the range of about 100,000 to about 200,000 g/mole.

In some embodiments, portions of the film may be textured, e.g. embossed, micro- or nano-perforated, to modify the disintegrability of the film.

As noted herein, the cells in the plurality of cells may differ one from the other in at least one property. Thus, in some embodiments, at least one portion of cells is different in at least one property from at least another portion of cells in the plurality of cells. The property may be at least one property selected from film thickness, molecular weight of the polymeric material, composition of the polymeric material, film texture, water solubility of the film, volume of cell, geometry of cell, geometry and size of disintegrable area, type of active agent contained therein, etc.

In order to maintain structural integrity of the device, the flexible substrate may be made of a material substantially non-disintegrable or non-soluble upon contact with a water-based fluid, e.g. perspiration or exudate. Thus, the substrate remains intact during use. The substrate may be made of any suitable material, e.g. a polymer, a natural fabric, a synthetic fabric, a woven flexible composite, etc. The device may further comprise additional external flexible layers, such as fabric layers, coatings, absorbing layers, hydrocolloids, and others.

As already noted, the cells are arranged in a spaced-apart manner. In some embodiments, the cells are spaced-apart by substantially non-disintegrable segments, thus also forming a peripheral seal around each cell. The non-disintegrable segments may have non-uniform thickness and/or density to permit flexibility and/or foldability of said segments. The segments may be perforated in order to afford further flexibility of the device. The non-disintegrable segments may be made of the same or different material as the flexible substrate.

The cells comprise at least one active agent for topical delivery to a target site one applied onto the application site on the skin. The active agent may be any agent, chemical or biological, intended to provide a therapeutic or cosmetic effect, that is required for delivery to the target site. The active agent may be in any suitable form, such as is in a form selected from a gel, a cream, an oil, an ointment, a non-aqueous liquid, a non-aqueous solution, an emulsion, a microemulsion, a powder, a flake, a granule, a microparticle, a microcapsule, a nanoparticle, a nanocapsule, a liposome, or any other suitable form. It is of note that the term also means to encompass formulations (i.e. pharmaceutical formulations or cosmetic formulations) and compositions comprising the active agent.

The pharmaceutical formulation may comprise the at least one active agent (substance, molecule, element, compound, entity, or a combination thereof) and at least one pharmaceutically acceptable carrier, for example, vehicles, adjuvants, excipients, or diluents. Such carriers are well-known to those who are skilled in the art.

The cosmetic formulation may comprise at least one active agent, e.g. a cosmetically-active agent, and at least one cosmetically acceptable carrier. The cosmetically-active agent may be capable of inducing, enhancing, arresting or diminishing at least one cosmetic non-systemic effect, and may be selected amongst dermatological agents, i.e. agents capable of inducing or modulating an effect on the skin of a subject, when administered in an effective amount. The cosmetically acceptable carrier may be selected from vehicles, adjuvants, excipients, and diluents, which are readily available to the public. The cosmetically acceptable carrier is typically selected to be chemically inert to the active agent or to any component thereof and one which has no detrimental side effects or toxicity under the conditions of use.

The choice of carrier, whether for cosmetic (non-systemic) or pharmaceutical formulations will be determined in part by the particular active agent. The cosmetic compositions or the pharmaceutical formulation may be formulated for topical, transepithelial, epidermal, transdermal, and/or dermal administration routes of the active agent. Accordingly, there is a wide variety of suitable formulations of the for active agents to be used in devices of the present disclosure. However, as parts of the device are designed to disintegrate upon contact with a water-based fluid, the carriers will be typically non-aqueous based. Thus, in some embodiments, the active agent may be formulated into a pharmaceutical or cosmetic composition that is essentially, at times entirely, devoid of water.

The pharmaceutical or cosmetic formulation may further comprise other standard additives such as emollients, moisturizers, thickeners, emulsifiers, neutralizers, coloring agents, fragrances, absorbers or filters, preservatives, gelling agents, fillers, sun screen agents, electrolytes, proteins, antioxidants and chelating agents.

Each cell may comprise one or more active agents, each having a similar or different activity. In some embodiments, all of the cells in the plurality of cells contain the same active agent.

In other embodiments, at least one portion of the cells contain an active agent differing from the active agent contained in at least another portion of cells. For example, a first portion of cells may contain an antiseptic, while a second portion of the cells (that is, for example, disintegrable at a slower rate) may contain an antibiotic. In another example, a first portion of cells that come into contact with the target site contain a first agent to be administered directly to the target site (for example an anti-burn active agent), while other cells that are positioned peripherally to the first portion contain a second agent to be administered to the peripheral area of the target site (for example a moisturizing agent or a hydrocolloid).

The segmentation into a plurality of cells may also permit delivering different active agents, which are unstable when combined or are not compatible for prolonged storage one with the other, to the same target site—as such different agents may be contained in adjacent spaced-apart cells. Due to the segmentation, such active agents are isolated one from the other during storage of the device, and only come into contact with one another when in use. Similarly, adjacent cells may contain different agents which react with one another upon contact to produce a desired active agent in situ at the target site.

The active agent may be selected from an anti-inflammatory agent, a non-steroidal anti-inflammatory (NSAID) agent, a pain-relief agent, wound healing promoting agents, an analgesic, an antihistamine, an opioid or opioid derivative, growth hormone, a cannabinoid, an antifungal agent, an antiviral agent, a coagulant, an antibiotic agent, a clotting factor, a clotting agent, a hemostatic agent, an antimicrobial agent, an antiseptic and a disinfectant, and others, as well as any mixture or combination thereof.

In some embodiments, the active agent may be selected from an anti-inflammatory agent, a pain-relief agent, wound healing promoting agents, an analgesic, an antihistamine, an opioid or opioid derivative, a cannabinoid, an antifungal agent, an antiviral agent, an antibiotic, and antimicrobial agent, an antiseptic, as well as any mixture or combination thereof.

Cannabinoids are a group of psychoactive and non-psychoactive compounds which have an activity on cannabinoid receptors in cells to repress neurotransmitter release in the brain. The term is meant to encompass cannabinoids which are obtained from natural sources by various processes of treatment or extraction, as well as to synthetically obtained cannabinoids. The cannabinoid may be selected from one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CDB), cannabidiol monomethylether (CBDM), cannabidiol-C₄ (CBD-C₄), cannabidivarinic acid (CBDVA), cannabidiorcol (CBD-C₁), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THCA-C₄), delta-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), delta-7-cis-iso-tetrahydrocannabivarin, delta-8-tetrahydrocannabinolic acid A (Δ⁸-THCA), delta-8-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahtdro-7-hydroxy-α-α-2-trimethyl-9-n-propyl-2,6-methano-2H-1-benzoxocin-5-methanol (OH-iso-HHCV), cannabiripsol (CBR), trihydroxy-delta-9-tetrahydroxycannabinol (triOH-THC), and any other cannabinoid or any mixture thereof.

In some embodiments, the cannabinoid is selected from CBD, CBDA, THC, and mixtures thereof.

At times, the disintegration of the cells needs to be accelerated, i.e. immediate release of the cell's content is desired. Therefore, such cells may be made of a highly-soluble polymeric material.

In another embodiment, the cells may contain, in addition to the active agent, a decomposable agent capable of forming a gaseous decomposition product upon contact with the fluid. In such cases, once initial disintegration of the cell occurs and the decomposable agent comes into contact with the fluid, it decomposes into voluminous gaseous decomposition products that cause abrupt rupturing of the cell and faster release of the active agent therefrom. Exemplary decomposable agents may be calcium bicarbonate (Ca(HCO₃)₂) or other relevant exothermic material.

According to some embodiments, the device may be a bandage, a dressing, a sleeve, a pad, an elastic pad, a standalone capsule, etc.

Such dressing utilities typically comprise one or more various layers of woven or non-woven fabric, e.g. elastic fabric, integrated one with the other to form the dressing. The devices of the present disclosure may be configured for association (e.g. adhering) or integration with the dressing utility to form an active dressing utility.

The device may further comprise an adhesive on at least a portion of the device's perimeter to enable temporary fixation and/or application of pressure onto the target site. Thus, in some embodiments, the device may be in the form of a plaster or an adherable bandage.

In another application, the device may be configured for insertion into a body cavity or lumen, such as an artery, a vein, intestinal tract, urinary tract, vaginally, rectally, etc. In such embodiments, the device may be configured to have a tubular form, for example, a tampon. In other such embodiments, the device may be configured for insertion into a wound, e.g. stab wound, gunshot wound, an ulcer, etc.

The term target site refers to a body organ to which delivery of the active agent is desired. The target site may be any body organ or tissue, including but not limited to, the skin, limbs and digits, abdomen, head and neck, internal organs (e.g. liver, heart, lungs, kidney, stomach, intestines and intestinal tract, pancreas, urinary tract, etc.). The term should be understood to denote any tissue of the subject, human or non-human, including the skin and the soft tissues of the body. The term also encompasses the blood.

The term organ refers to any part of the body of an animal or of a human that is capable of performing some specialized physiological function. The term may include any part of such an organ or a collection of one or more of such organs. Non-limiting examples of organs include the heart, lungs, kidney, ureter, urinary bladder, adrenal glands, pituitary gland, skin, prostate, uterus, reproductive organs, liver, gall-bladder, brain, spinal cord, stomach, intestine, appendix, pancreas, lymph nodes, breast, salivary glands, lacrimal glands, eyes, spleen, thymus, bone marrow, etc.

As noted the delivery of the active agent to the target site is by topical delivery or delivery to or through the skin layers. Hence, in such embodiments, the devices may be suitable for external application, e.g. to treat a skin injury or external muscular or vascular injury (such as wounds, cuts, punctures, large surface hemorrhaging injuries, etc.). Other topical applications may include treatment of skin lesions, burns of various degrees (e.g. chemical burns, thermal burns, electrical burns, radiation burns), skin infections (e.g. bacterial, fungal or viral infections), skin ulcers, etc.

As known, human skin is made of numerous layers which may be divided into three main group layers: Stratum corneum which is located on the outer surface of the skin, the epidermis and the dermis. While the Stratum corneum is a keratin-filled layer of cells in an extracellular lipid-rich matrix, which in fact is the main barrier to drug delivery into skin, the epidermis and the dermis layers are viable tissues. The epidermis is free from blood vessels, but the dermis contains capillary loops that can channel therapeutics for transepithelial systemic distribution. The term topical as used herein refers to the application of a device directly onto at least a portion of a subject's skin (human or non-human skin) so as to achieve a desired effect at a target site by delivering the active agent directly to a skin target site or to a target site in an organ different from the skin by transdermal delivery through various layers of the skin. In some embodiments, the desired effect is achieved at the target site without inducing one or more systemic effects. In other embodiments, the active agent delivered by the device induces at least a partial systemic effect which contributes to the induction of at least one desired effect at the target site.

The application device described herein may also be used in order to deliver the active agent into the external skin of the subject, the eye or skin areas surrounding the eye, or to internal skin portions, e.g. to a body cavity, body lumen, body tissue or a tubular organ. For example, the application device may be used to deliver the active agent by inserting the device into a cavity, lumen, stab wound, gunshot wound, or tubular organ (e.g. a vein, an artery, a bile duct, respiratory tract, gastrointestinal tract, urinary tract, fallopian tubes, etc.) and delivering the carriers directly to the target site within the organ. Thus, in some embodiments, the application device may be configured for insertion into a body cavity or lumen.

As noted above, the device may comprise a plurality (two or more) cells that differ in their disintegration rate, such that the difference in the integration rate may be utilized in order to provide a sequence of administration of a plurality of doses of the active agent. By controlling the rate of disintegration, the device may be utilized for controlled release multiple sequential doses of the active agent, the intervals between the doses being determined by the difference in the disintegration rate of the cells.

Thus, another aspect provides a flexible device for topical delivery of a sequence of doses of at least one active agent to a target site, the device comprises a flexible substrate for placing onto a skin portion and having a plurality of spaced-apart cells, each cell in said plurality containing an effective dose of said at least one active agent and having at least one wall portion made of a film of at least one polymeric material that is at least partially disintegrable upon contact with an aqueous fluid to thereby release the active agent to the target site, wherein the cells differ in their disintegration rate, such that the difference in the disintegration rate forms a sequence of disintegration of the cells with a defined time interval between disintegration of subsequent cells in said sequence, the time interval being defined by the difference in disintegration rate.

In another aspect, there is provided a method of manufacturing a flexible device for topical delivery of at least one active agent to a target site (e.g. the flexible device described herein), the method comprising:

forming a plurality of spaced-apart cells each cell having at least one wall portion made of a film of at least one polymeric material that is at least partially disintegrable upon contact with an aqueous fluid (perspiration, exudate, lacrimal fluid, or externally-applied water-based fluid);

filling said cells with said at least one active material; and

sealing the cells with a flexible substrate to form the device.

According to some embodiments, the plurality of spaced-apart cells may be formed by any method known to a person of skill that enables shaping of the polymeric film into spaced-apart cells (e.g. casting, extrusion, heat-forming, vacuum-forming, etc.). Once the empty cells are obtained, the cells are filled with the active agent and then sealed by the flexible substrate to result in the flexible device.

Another aspect of this disclosure provides a method of manufacturing a device as described herein, the method comprises:
(a) bringing a flexible substrate and a film of at least one aqueous fluid-disintegrable (perspiration-disintegrable, exudate-disintegrable, or lacrimal fluid-disintegrable, or externally applied water-based fluid-disintegrable) polymeric material in proximity one to the other;
(b) integrating said flexible substrate with said film to form a plurality of spaced-apart pre-cells, the pre-cells having a portion of their perimeter non-integrated,
(c) introducing at least one active agent into said pre-cells through the non-integrated portion, and
(d) sealing said pre-cells by integrating said flexible substrate with said film along said portion to thereby form said spaced-apart cells.

Thus, the method includes first preparing spaced-apart pre-cells, which have a peripheral opening (i.e. the pre-cells are not sealed in at least a portion of their periphery). The pre-cells are filled with the active agent or a composition comprising an active agent, and then the peripheral opening is sealed to form the cells. This sequence of steps, namely steps (a) to (d), may be repeated to manufacture a device comprising an array of said spaced-apart cells.

At times, step (d) of one cycle of the method may also be step (b) of the subsequent cycle of the method. Namely, sealing the pre-cells in step (d) of a first cycle may also form the next in-line pre-cells. Thus, in a single integration, both already prepared pre-cells are sealed and the next in-line pre-cells in the array are formed.

Integration means bringing the substrate and polymeric film into intimate contact and applying conditions to adhere them one to the other. Integration may, by some embodiments, be carried out by welding, such that the film and the substrate are adhered one to the other along defined welding zones. Welding may be carried out by any suitable welding technique, for example heat sealing, contact welding, high frequency welding, ultrasonic welding, laser welding, solvent welding and others.

The welding thus forms said plurality of cells that are spaced-apart by substantially non-disintegrable segments constituted by the welding zones. In some embodiments, said segments are non-uniform in thickness and/or density to permit flexibility and/or foldability of said segments after integration. In other embodiments, the method may further include a step (e), in which the non-disintegrable segments are perforated in order to permit further flexibility/foldability of said segments.

The perimeter of the cells (i.e. the interface between the cell and the non-disintegrable segments surrounding it) may have various profiles that may be obtained by modifying the parameters of the welding conditions. Such changes in profile of the interface may be utilized to control, for example, the rupturability of the cells.

In some embodiments, the film of the polymeric material may have pre-defined cell-forming sections and seal-forming sections, such that integrating is carried out by welding said film to said substrate along said seal-forming sections. In such cases, said cell-forming sections may be formed from a disintegrable polymeric material, while the seal-forming sections may comprise or formed of a non-disintegrable material (e.g. a non-disintegrable polymeric material). The seal-forming sections may also comprise a laminate of polymeric layers having different disintegration properties.

The method may further comprise a step prior to (a) of texturing the film or the cell-forming sections of said film. Texturing may include embossing, micro- or nano-perforating the film, thereby forming weaker spots in the cell to promote faster disintegration.

The device may be utilized as a stand-alone device, or alternatively as an active component in a dressing assembly (e.g. an elastic bandage). Therefore, the methods described above may further comprise a step of associating or integrating the device with at least one fabric layer, e.g. an elastic fabric layer or any other suitable dressing fabric (for example woven cotton, gauze, etc.).

In another aspect, the present disclosure provides a method of topically delivering at least one active agent to a target site of a subject, comprising contacting the flexible device described herein with a skin portion of the patient, such that at least a portion of the plurality of cells comes into contact with an aqueous fluid (perspiration, exudate, lacrimal fluid, or externally applied water-based fluid) to cause selective disintegration of cells for releasing said active agent for delivery to the target site.

In some embodiments, contacting with said fluid causes (i) a portion of said cells to disintegrate and release a first active agent to the target site, followed by (ii) disintegration of another portion of cells for releasing a second active agent to the target site.

In other embodiments, contacting with the fluid causes all of the cells that are brought into contact with the target site to disintegrate.

In another aspect, there is provided a method of treating a disease, condition or disorder, comprising contacting a flexible device of this disclosure with a skin portion of a patient, at least a portion of cells in the device comprise at least one active agent for treating said disease, condition or disorder, said cells being selectively disintegrable upon contact with an aqueous fluid (e.g. perspiration, exudate, lacrimal fluid or an externally-applied water-based fluid) to release said active agent to said skin portion.

In another aspect, there is provided a method of treating a skin infection, skin condition or skin disorder, comprising contacting a flexible device of this disclosure with an infected skin portion of a patient, at least a portion of cells in the device comprise at least one active agent for treating said skin infection, said cells being selectively disintegrable upon contact with an aqueous fluid as defined herein to release said active agent to said infected skin portion.

A further aspect provides a method of treating a skin burn, comprising contacting a flexible device of this disclosure with said skin burn, at least a portion of cells in the device comprises at least one active agent, said cells being selectively disintegrable upon contact with an aqueous fluid defined herein to release said active agent to said skin burn.

Yet another aspect provides a method of topically delivering an anti-inflammatory agent to a target site, comprising contacting a flexible device as herein described with a skin portion of a patient, at least a portion of cells in the device comprise at least anti-inflammatory agent, said cells being selectively disintegrable upon contact with an aqueous fluid as defined herein to release said anti-inflammatory agent to said skin portion.

A further aspect provides a method of managing pain by topical delivery of at least one active agent (e.g. an analgesic), comprising contacting a flexible device of this disclosure with a skin portion of a patient, at least a portion of cells in the device comprise said at least one active agent, said cells being selectively disintegrable upon contact with an aqueous fluid as defined herein to release said active agent to said skin portion.

In another aspect, there is provided a method of treating a skin ulcer or a pressure ulcer (or pressure wound), comprising contacting a flexible device of this disclosure with said ulcer or wound, at least a portion of cells in the device comprise at least one active agent, said cells being selectively disintegrable upon contact with an aqueous fluid as defined herein to release said active agent to said skin ulcer or wound.

In yet another aspect, there is provided a method of delivering an active agent to the eye, comprising contacting a flexible device of this disclosure with an eye or a skin area surrounding the eye of a patient, at least a portion of cells in the device comprise said at least one active agent, said cells being selectively disintegrable upon contact with an aqueous fluid as defined herein to release said active agent to the eye.

In a further aspect, there is provided a method of topically delivering a sequence of doses of at least one active agent to a target site, comprising contacting a flexible device as described herein with a skin portion of a patient, such that the plurality of cells come into contact with an aqueous fluid as defined herein to cause the cells to disintegrate in a sequence of disintegration with a defined time interval between disintegration of subsequent cells in said sequence, the time interval being defined by the difference in disintegration rate, for topically releasing a sequence of doses of said active agent to said target site.

It is to be understood that the devices are designed to release the active agent when contacted with an aqueous fluid. Namely, the active agents may be released from the device upon contact with an aqueous bodily fluid (e.g. perspiration, exudate, lacrimal fluid, etc.), or when externally applying a water-based fluid onto the device during and/or after application of the device to the skin.

According to some embodiments, the active agent is topically delivered to the target site (whether the target site is the skin or a different organ) in an effective amount to cause at least one therapeutic effect at the target site. As known, the effective amount for purposes herein may be determined by such considerations as known in the art. The amount applicable to achieve the desired therapeutic effect, depends, inter alia, on the type and severity of the condition to be treated and the treatment regime. The effective amount is typically determined in appropriately designed clinical trials (dose range studies) and the person versed in the art will know how to properly conduct such trials in order to determine the effective amount.

The active agent may be used to induce at least one effect, e.g. a therapeutic effect; namely, the active agent is capable of inducing, enhancing, arresting or diminishing at least one effect, by way of treatment or prevention of unwanted conditions in a subject. The at least one active agent (substance, molecule, element, compound, entity, or a combination thereof) may be selected amongst therapeutic agents, i.e. agents capable of inducing or modulating a therapeutic effect when administered in a therapeutically effective amount, and non-therapeutic agents, i.e. which by themselves do not induce or modulate a therapeutic effect but which may be combined with another agent to endow the desired therapeutic effect.

Treatment or any lingual variation thereof, as used herein, refers to the administering of a therapeutic amount of the active agent or composition comprising it, which is effective to ameliorate undesired symptoms associated with a condition in a subject, to prevent the manifestation of such symptoms before they occur, to slow down the progression of the condition, slow down the deterioration of symptoms, to enhance the onset of remission period, slow down the irreversible damage caused in the progressive chronic stage of the condition, to delay the onset of said progressive stage, to lessen the severity or cure the disease, to improve survival rate or more rapid recovery, or to prevent the condition from occurring or a combination of two or more of the above.

The term subject as used herein refers to human and non-human subjects (mammals), i.e. the devices may be utilized in treating humans or for veterinary purposes.

As used herein, the term about is meant to encompass deviation of ±10% from the specifically mentioned value of a parameter, such as thickness, time, molecular weight, etc.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A shows a schematic cross-sectional view of a flexible device according to an embodiment of this disclosure.

FIG. 1B is a schematic representation of a dressing utility comprising an exemplary flexible device according to an embodiment of this disclosure.

FIGS. 2A-2C show schematic representations of flexible devices according to some embodiments of the present disclosure.

FIG. 3 shows another schematic representation of a flexible device according to this disclosure, comprising cells with variable height profiles.

FIG. 4A-4B are schematic representations of an adhesive plaster comprising the flexible device according to an embodiment of the present disclosure with a protective layer covering the device (FIG. 4A) and with the protective layer partially removed from the device for application of the device onto a target site (FIG. 4B).

FIG. 5 shows another schematic representation of a flexible device according to this disclosure.

FIGS. 6A-6C are schematic steps of a manufacturing method of a flexible device according to an embodiment of this disclosure.

FIGS. 7A-7C are schematic steps of a manufacturing method of a flexible device according to another embodiment of this disclosure.

FIG. 8 shows a device according to another embodiment of this disclosure, including formed weak areas in the cells for directed disintegration of the polymeric material.

### DETAILED DESCRIPTION OF EMBODIMENTS

As noted above, the present disclosure concerns devices that are designed to topically deliver one or more active agents to a target site in controlled and selective manner and sequence of delivery, while having improved flexibility to maximize the contact area with the skin portion to which the device is applied. Some non-limiting examples will now be described in order to demonstrate how the devices of this disclosure maybe designed and manufactured.

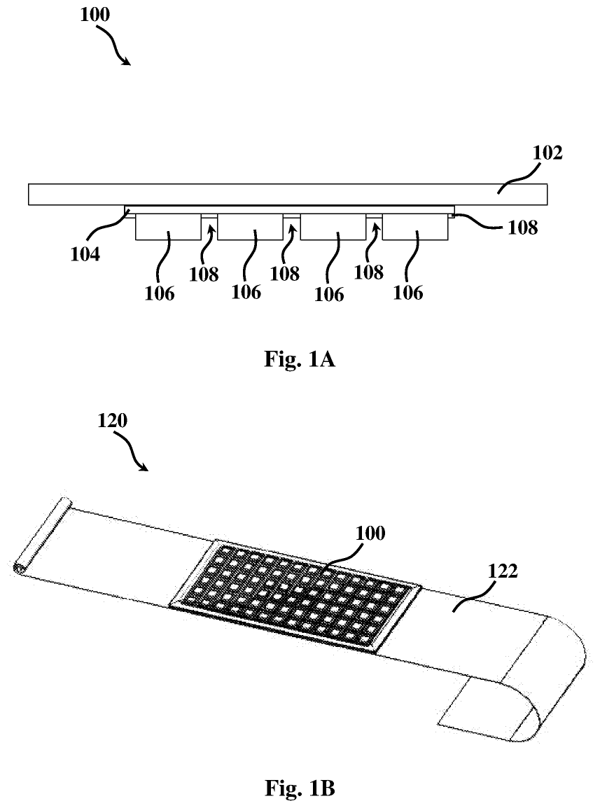

FIG. 1A is a schematic cross-sectional view of a flexible device 100, such as a personal bandage, that comprises an elastic woven fabric 102, onto which flexible substrate 104 is fixated. Fixation may be obtained by adhering, stitching, or any other suitable means. The flexible substrate 104, which may be made, for example, from a non-disintegrable polymeric sheet, carries a plurality of cells 106, made from a film of a polymeric material 108. The cells 106 are made of a perspiration- or exudate-disintegrable material, typically a perspiration- or exudate-disintegrable polymer (at times a lacrimal fluid disintegrable polymer), and contain therein at least one active compound. The cells 106 are spaced-apart by non-disintegrable segments 110, which may or may not be perforated or scored. Due to the segmentation to a plurality of cells (as well as providing, in some cases, perforated or scored non-disintegrable segments), improved flexibility of the device is obtained, as the device bendable and/or foldable along the non-disintegrable segments 110. Such flexibility enables improved contact with the skin portion onto which the device is applied. For example, when applying the device onto a limb (leg or arm), such flexibility allows the device to conform to the contours of the limb and improve contact therebetween for effective topical delivery of the active agent to the target site.

A dressing utility 120, e.g. an elastic bandage, comprising device 100 is shown in FIG. 1B. The dressing utility 120 includes at least one layer of fabric 122, onto which device 100 is integrated. The dressing utility may be packed as individual or personal dressings (e.g. pre-packaged personal bandage) or be formed as a strip (e.g. a rolled continuous strip) that can be cut by the user on-site to a desired length.

It is to be noted that although the examples provided herein show rectangular cells, having similar geometry and size, the cells may not be necessarily so. A variety of shapes and sizes of cells may be utilized, depending on various considerations, such as rupturability, method of manufacture, targets delivery site, etc.

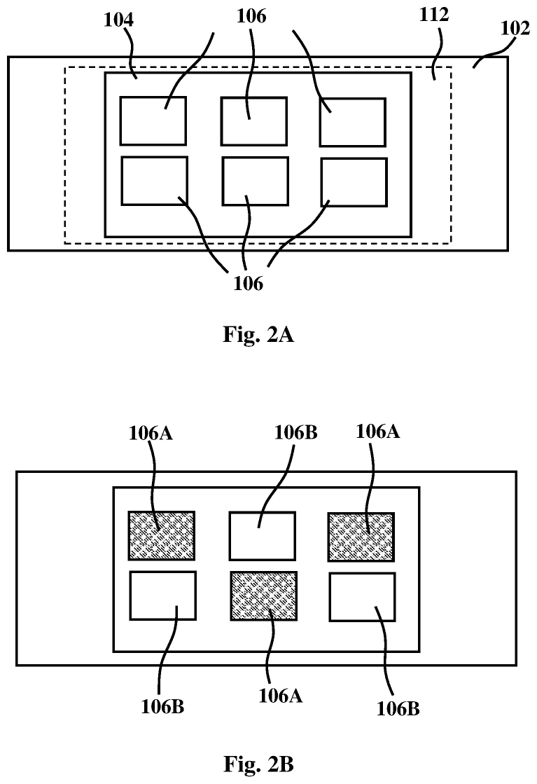

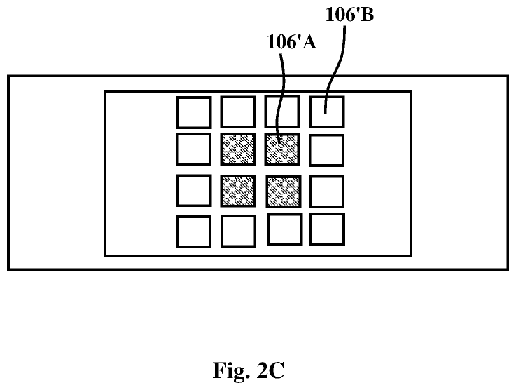

FIG. 2A shows another schematic device, similar to that of FIG. 1A. In this device, a peripheral section of the substrate 112 may include an adhesive, such that once applied, the device is adhered to the skin. For example, the device may be a plaster that adheres to the skin or a patch to be applied to an eye.

In the device of FIG. 2B, a portion of cells 106A differs in at least one of its properties from another portion of cells 106B. The cells may differ, for example, in one or more of the film thickness, the molecular weight of the polymeric material, the composition of the polymeric material, the film texture, water solubility of the film, the volume of cell, the geometry of cell, the size of disintegrable area, the type of active agent contained within the cell, and others.

Another exemplary arrangement is shown in FIG. 2C, in which a portion of cells 106′A are located at a center of the substrate, while another portion of the cells 106′B is located at the substrate's periphery. For example, cells 106′A come into contact with the target site and contain a first agent to be administered directly to a target site on the skin (e.g. a burn), while cells 106′B that are positioned peripherally and contain a second agent to be administered to the peripheral area of the target site (e.g. the skin tissue surrounding the burn area).

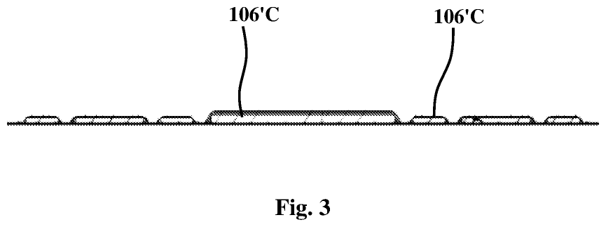

FIG. 3 shows another example of a flexible device of this disclosure. In this example, the device comprises cells 106′C and 106′D, differing in their height and volume. Such variance in volume enables controlling the quantity of active agent that is contained, and subsequently released, from the cells. For example, in some instances it is desired that a first active agent contained in cell 106′C is delivered to the target site in a larger quantity than a second active agent contained in cell 106′D. The variance in height of the cells can also vary the timing of contact of each cell with the target site. Namely, as cell 106′C has a larger height (when measuring from the base of the device) than cell 106′D, cell 106′C will contact the target site first to release the active agent contained therein, and only thereafter cell 106′D, which has a smaller height, will contact the target site. This is another mechanism by which timing of release of a sequence of active agents may be obtained by the device.

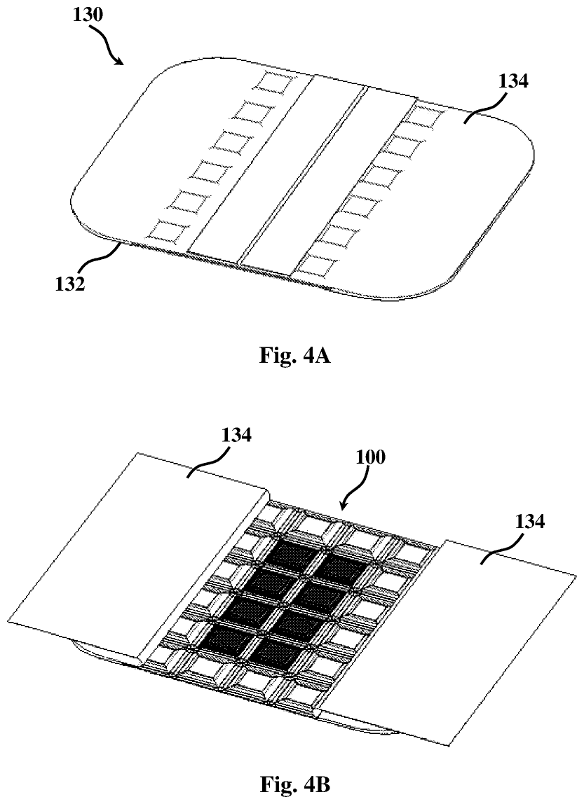

FIGS. 4A-4B show an exemplary adhesive plaster 130 comprising the device 100. The adhesive plaster 130 comprises a fabric carrier layer 132, onto device 100 is fixed. An adhesive is typically applied on the area not covered by device 100, to permit adhering the plaster during use over a skin portion. During storage a protective layer 134 covers the device and the adhesive area, as seen in FIG. 4A; before application onto the skin, the protective layer 134 is removed from the device (partial removal is seen in FIG. 4B), thus enabling exposure of device 100 and adhering plaster 130 onto the skin.

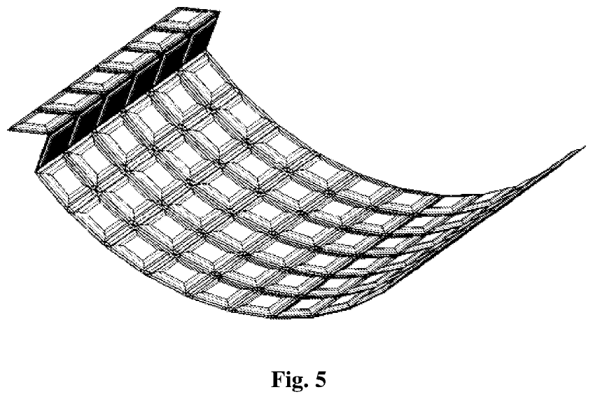

As noted above, the segmentation of the device into discrete, spaced-apart cells renders the device with improved flexibility, as shown in FIG. 5. Such flexibility enables the device to conform in shape to irregular surfaces (such as a limb) and ensure contact with the skin for maximizing contact with the skin and hence improving topical delivery of the active agent to the target site.

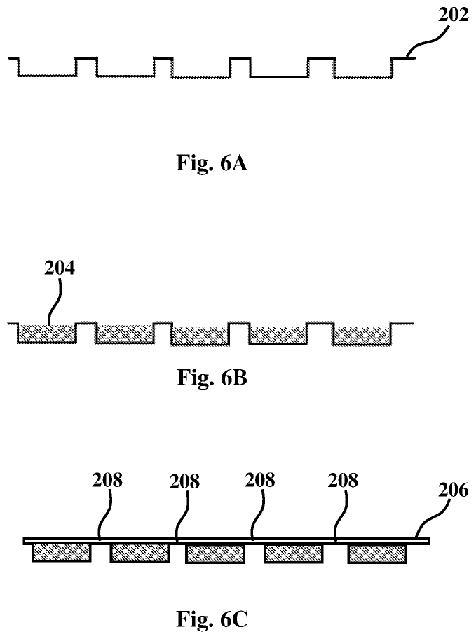

FIGS. 6A-6C show a schematic illustration of a method of producing flexible devices of this disclosure. Shown in FIG. 6A is a disintegrable polymeric film 202, shaped into a plurality of spaced-apart cells. As noted above, the polymeric film may be shaped into cells as a first step of production, or may be a priori provided in a shaped form. Shaping may be carried out by any suitable method known to a person of skill. The cells are filled with at least one active agent 204, as shown in FIG. 6B, and in the next production step (FIG. 6C) the flexible substrate 206 is integrated with the polymeric film at designated integration segments 208 to seal the cells, and thereby form the device. Additional production steps may include attaching the device to other elements, such as a flexible fabric.

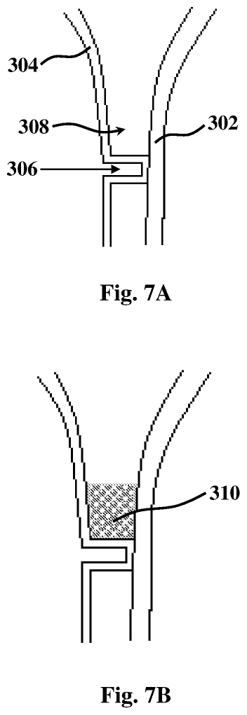

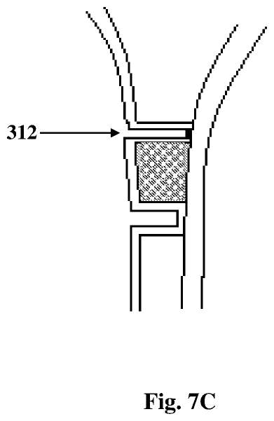

Another manner of manufacturing is shown schematically in FIGS. 7A-7C, in which the substrate 302 is brought into proximity with the disintegrable polymeric film 304. The substrate and the film are integrated, e.g. by welding, along a portion of the pre-cell perimeter 306 to form open pre-cells 308 (FIG. 7A), into which the active agent 310 may be filled (FIG. 7B). After filling, the pre-cells are sealed by integrating the remainder of their perimeter 312, e.g. by welding, to form the cells (FIG. 7C).

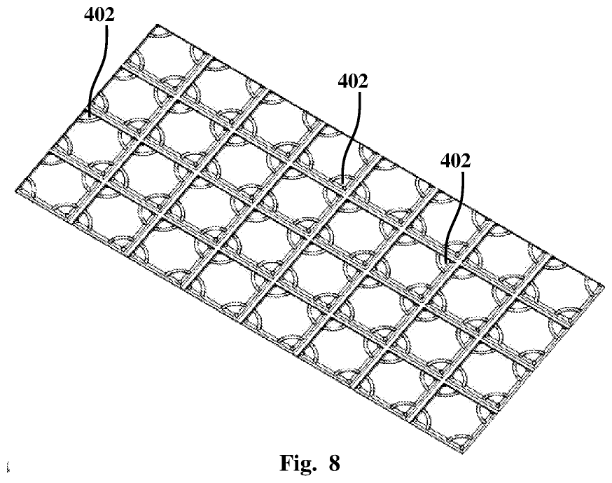

The manufacturing process of the device may comprise various other stages in order to form weak areas or spots in the cell, such weak spots function as areas for initiating disintegration of the polymeric material once in contact with perspiration, exudate or lacrimal fluid at the skin portion onto which the device is applied. For example, cells may be treated by laser treatment in order to form areas of reduced thickness of the polymeric film forming the cells at various locations on the cell. As seen in FIG. 8, laser treatment, or any other suitable abrasive method, is used to form weak areas at the corners 402 of the cells. Similarly, weak spots may be formed at any location on the surface of the cell, e.g. in the center of the cell's area (not shown). It is noted that the weak spots are not necessarily obtained by locally reducing the thickness of the polymeric film, but may also be formed by other means, such as local changes in the polymeric structure or even by using a different polymeric material to form the weak spots.

Alternatively, or in addition, embossing may be used on the entire surface of the cell or at selected portions of the surface of the cell to provide further local weakening to direct the disintegration of the cell to the desired location.

As a person of the art would appreciate, the devices provided in the Figures are mere exemplary devices, and it is to be understood that other devices falling within the scope of the claims are also encompassed by this disclosure.

1. A flexible device for topical delivery of at least one active agent to a target site, the device comprises a flexible substrate for placing onto a skin portion and having a plurality of spaced-apart cells, each cell in said plurality containing at least one active agent and having at least one wall portion made of a film of at least one polymeric material that is at least partially disintegrable upon contact with an aqueous fluid to thereby release the active agent to the target site.

2. The device of claim 1, wherein said aqueous fluid is selected from the group consisting of perspiration, exudate, and lacrimal fluid.

3.-4. (canceled)

5. The device of claim 1, wherein the aqueous fluid is a water-based fluid applied externally onto the device during and/or after application to the skin portion.

6. The device of claim 1, wherein the cells in said plurality of cells are configured for selective disintegration upon contact with said aqueous fluid.

7. The device of claim 6, wherein at least one portion of cells being different in at least one property from at least another portion of cells in said plurality of cells, said property being at least one property selected from film thickness, molecular weight of the polymeric material, composition of the polymeric material, film texture, water solubility of the film, volume of cell, geometry of cell, size of disintegrable wall portion, and type of active agent contained therein.

8. (canceled)

9. The device of claim 1, wherein said polymeric material is selected from polysaccharide, polyethyleneoxide (PEO), polyvinyl-pyrrolidone (PVP), polyvinyl-alcohol (PVA), polyacrylic acid (PAA), polyacryloamides, polyoxazoline, cellulose ethers.

10.-12. (canceled)

13. The device of claim 1, wherein said at least one active agent is selected from an anti-inflammatory agent, a pain-relief agent, wound healing promoting agents, an analgesic, an antihistamine, an opioid or opioid derivative, growth hormone, a cannabinoid, an antifungal agent, an antiviral agent, an antiseptic, an antimicrobial agent, an antibiotic, and a disinfectant.

14.-15. (canceled)

16. The device of claim 1, wherein all of the cells in said plurality of cells contain the same active agent and the cells differ in their disintegration rate, such that the difference in the integration rate forms a sequence of disintegration of the cells with a defined time interval between disintegration of subsequent cells in said sequence, the time interval being defined by the difference in the disintegration rate.

17.-20. (canceled)

21. The device of claim 1, wherein said polymeric material is fully disintegrable within between about 5 second and 30 minutes from contact with said perspiration or exudate.

22.-25. (canceled)

26. The device of claim 1, wherein said flexible substrate being made of a material substantially non-disintegrable upon contact with aqueous fluid.

27.-28. (canceled)

29. The device of claim 1, being a bandage, a dressing, or a sleeve.

30. The device of claim 29, further comprising an adhesive on at least a portion of the device's perimeter.

31. (canceled)

32. The device of claim 1, being configured for insertion into a body cavity or lumen.

33.-36. (canceled)

37. A method of manufacturing a device of claim 1, the method comprising:
(a) bringing a flexible substrate and a film of at least one aqueous fluid-disintegrable polymeric material in proximity one to the other;
(b) integrating said flexible substrate with said film to form a plurality of spaced-apart pre-cells, the pre-cells having a portion of their perimeter non-integrated,
(c) introducing at least one active agent into said pre-cells through the non-integrated portion, and
(d) sealing said pre-cells by integrating said flexible substrate with said film along said portion to thereby form said spaced-apart cells.

38. The method of claim 37, wherein steps (a) to (d) are repeated to manufacture a device comprising an array of spaced-apart cells.

39. (canceled)

40. The method of claim 37, wherein said integrating forms a plurality of cells that are spaced apart by substantially non-disintegrable segments.

41. (canceled)

42. The method of claim 37, wherein said film having cell-forming sections and seal-forming sections, such that integrating is carried out by welding said film to said substrate along said seal-forming sections.

43. The method of claim 42, wherein said cell-forming sections are formed from a disintegrable polymeric material.

44. The method of claim 42, wherein said seal-forming sections comprise or formed of a non-disintegrable polymeric material, or said seal-forming sections comprise a laminate of polymeric layers having different disintegration properties.

45. (canceled)

46. The method of claim 37, wherein the cells are spaced apart by substantially non-disintegrable segments constituted by integrated seal-forming sections, said segments being non-uniform in thickness and/or density to permit flexibility and/or foldability of said segments after integration.

47. (canceled)

48. The method of claim 37, further comprising a step prior to (a) of texturing said film or cell-forming sections of said film.

49. (canceled)

50. The method of claim 37, further comprising associating the device with at least one fabric layer.

51. (canceled)

52. A method of topically delivering at least one active agent to a target site, comprising contacting a flexible device of claim 1 with a skin portion of a patient, such that at least a portion of the plurality of cells comes into contact with an aqueous fluid to cause selective disintegration of cells for topically releasing said active agent to said target site.

53. The method of claim 52, wherein contacting with said aqueous fluid (i) a portion of said cells to disintegrate and release a first active agent to the target site, followed by (ii) disintegration of another portion of cells for releasing a second active agent to the target site.

54.-63. (canceled)

64. The method of claim 52, wherein said active agent is selected from the group consisting of an active agent for treating skin infection, an active agent for treating a skin burn, an anti-inflammatory, an active agent for treating pain, an active agent for treating a skin ulcer or a pressure ulcer.
